(19) United States
(12) Patent Application Publication
(10) Pub. No.: US 2003/0184711 A1
(43) Pub. Date: Oct. 2, 2003

(54) Slit lamp microscope

(76) Inventors: Tomoyoshi Abe, Tokyo (JP); Yasufumi Fukuma, Tokyo (JP); Yutaka Yoneda, Tokyo (JP)

Correspondence Address: ARMSTRONG, WESTERMAN & HATTORI, LLP, Suite 1000, 1725 K Street, N.W., Washington, DC, 20006, US

(21) Appl. No.: 10/396,721

(22) Filed: Mar. 26, 2003

(30) Foreign Application Priority Data
Mar. 29, 2002 (JP) 2002-95114

(51) Int. Cl. A61B 3/10

(52) U.S. Cl. 351/214

(57) ABSTRACT

There is provided a slit lamp microscope in which a dedicated image pickup device is installed to an observation system to enhance the image pickup function for an eye to be examined, operability of the observation system by an examiner is excellent, and moreover the cables are not exposed, thereby achieving a superior outward appearance. The slit lamp microscope includes an illumination system for illuminating an eye to be examined, an observation system for observing the eye to be examined, a support arm for supporting the observation system on the upper end side thereof, a base for rotatably on a protruding shaft barrel portion thereof the illumination system and the lower end side of the support arm supporting the observation system, a pedestal for supporting the base, a chin rest stand for securing the eye to be examined with a state in which the eye is opposed to the observation system, and a table on which the pedestal is installed and to which the chin rest stand is attached. An image pickup device composed of an image pickup unit for taking an observation image of the eye to be examined from the observation system and an operation processing unit for conducting image pickup operation and image signal processing, which has an operation portion and is integrally formed with the image pickup unit, is attached to the support arm in a position near the observation system, and an operation pressing button group is provided in a direction orthogonal to an end of operation processing means along a rotation under line of the support arm.

### BACKGROUND OF THE INVENTION

1. Field of the Invention

The present invention relates to a slit lamp microscope used in the field of ophthalmology, and more particularly to a slit lamp microscope in which an image pickup function with respect to an eye to be examined is enhanced and whose operability is excellent.

2. Description of the Related Art

Up to now, there has been used a slit lamp microscope as a kind of ophthalmologic apparatus used in the field of ophthalmology.

According to such a slit lamp microscope, slit-like illumination light (that is, slit light) and background illumination light for illuminating the entire eye to be examined are irradiated from an illumination system supported to a pedestal installed on a table to an eye to be examined which is secured by putting the chin of the subject on a chin rest stand arranged upright on an end of the table, and a slit image of the eye to be examined (for example, a cornea cross sectional image) is observed by an observation system which is supported on the pedestal through a support arm so as to be rotatable in a horizontal direction.

Then, on the optical path of a binocular microscope type observation system that is attached to the support arm and is operated to rotate, the slit image and the entire image (for example, a still image) of the eye to be examined are taken using an image pickup device such as an externally attached digital camera attached through a camera attachment. The taken image information is transferred to an image processing device and the like using cables. Thus, image processing and image display are conducted by the image processing device to allow observation by an examiner.

However, up to now, image pickup devices such as digital cameras are not specifically designed for a slit lamp microscope. Thus, in conventional slit lamp microscopes, generally, a digital camera with a large weight which is commercially available or for general purpose use is attached to the observation system through the camera attachment. Thus, the digital camera is exposed in the peripheral portion of the observation system while being protruded outside.

Further, the camera attachment is constructed such that an observation light flux is led from one of the optical paths of the binocular microscope type observation system to a digital camera side using a beam splitter. Thus, it is difficult to attach the camera attachment and the digital camera to the observation system with a good weight balance.

On the other hand, the support arm for supporting the observation system forms a cantilever support structure in which, due to the structure of the slit lamp microscope, a lower end portion is supported by a bearing portion provided on the pedestal, a middle portion is curved to rise from a horizontal direction to a vertical direction, and the observation system is attached to the top end side.

As a result, when the camera attachment and the digital camera are attached, the weight load balance achieved with respect to the support arm that rotates about the lower end portion as a support shaft is significantly disturbed. Therefore, there is a problem in the operability in rotating the observation system (such necessary rotational operation force is significantly increased as compared with the case where the digital camera is not attached).

Also, according to a conventional structure in which the digital camera is attached to the observation system through the camera attachment, the cables necessary to operate the digital camera are surely exposed to the vicinity of the observation system. Thus, when an examiner rotates the observation system in a horizontal direction to change an observation position for the eye to be examined, there is a problem generated in that the exposed cables come into contact with the observation system, thereby reducing operability for the examiner.

Further, there is a problem in that the slit lamp microscope as a whole gives a rather disorderly appearance due to the exposed routing of the cables.

### SUMMARY OF THE INVENTION

The present invention has been made in view of the above circumstances. Therefore, an object of the present invention is to provide a slit lamp microscope in which an image pickup function for an image of an eye to be examined is enhanced by installing a dedicated image pickup device having a small size and a novel structure to the observation system, operability of the observation system by an examiner is excellent, and moreover the cables are not exposed, thereby also achieving a superior outward appearance.

In order to attain the above object, according to a first aspect of the present invention, the invention relates to a slit lamp microscope including: an illumination system for illuminating an eye to be examined; an observation system for observing the eye to be examined; a support arm for supporting the observation system on the upper end side thereof; a base for rotatably supporting on a protruding shaft barrel portion thereof the illumination system and the lower end side of the support arm supporting the observation system; a pedestal for supporting the base; a chin rest stand for securing the eye to be examined with a state in which the eye to be examined is opposed to the observation system; and a table on which the pedestal is installed and to which the chin rest stand is attached, the slit lamp microscope being characterized in that an image pickup device composed of an image pickup unit for taking an observation image of the eye to be examined from the observation system and an operation processing unit for conducting image pickup operation and image signal processing, which has an operation portion and is integrally formed with the image pickup unit, is attached to the support arm in a position near the observation system, and an operation pressing button group is provided in a direction orthogonal to an end surface of the operation processing unit along a rotation center line of the support arm.

According to a second aspect of the present invention, in the first aspect of the invention, the slit lamp microscope is characterized in that an end surface of the operation portion of the operation processing unit is arranged in a direction orthogonal to an eyepiece optical path of the observation system.

According to a third aspect of the present invention, in the first or second aspect of the invention, the slit lamp microscope is characterized in that elements which constitute the image pickup unit and the operation processing unit with the operation portion of the image pickup device are incorporated in an image pickup device main body such that the elements are arranged in a balanced manner in weight with respect to a center line of the support arm.

According to a fourth aspect of the present invention, in any one of the first through third aspects of the invention, the slit lamp microscope is characterized by further comprising an accommodating groove provided in the support arm, a cable hole provided in the protruding shaft barrel portion of the base which protrudes from the pedestal to the chin rest stand side, a cable hole provided in the chin rest stand, and a cable hole provided in the table, which together form a routing path for leading cables under the table, that are to be connected with the image pickup device.

According to the first through fourth aspects of the present invention, there can be provided a slit lamp microscope in which the image pickup function for the eye to be examined can be improved by installing the dedicated image pickup device, the rotating operation of the observation system can be conducted in a satisfactory manner, and the cables are not exposed in the vicinity of the support arm so that the operability of the observation system by an examiner is excellent and also a superior outward appearance is achieved.

Also, even when the observation system is rotated in an arbitrary direction about the support arm as a rotation center, an examiner is always opposed to the front side of an operation portion and can cause an operating force to the operation button group to always act in the direction of the rotation center of the support arm. Thus, the operability of the operation portion by an examiner can be improved.

According to a fifth aspect of the present invention, the invention relates to a slit lamp microscope including: an illumination system for illuminating an eye to be examined; an observation system for observing the eye to be examined; a support arm for supporting the observation system on the upper end side thereof; a base for rotatably supporting on a protruding shaft barrel portion thereof the illumination system and the lower end side of the support arm supporting the observation system; a pedestal for supporting the base; a chin rest stand for securing the eye to be examined with a state in which the eye to be examined is opposed to the observation system; and a table on which the pedestal is installed and to which the chin rest stand is attached, the slit lamp microscope being characterized by including an image pickup device composed of: an image pickup unit for taking an observation image of the eye to be examined from the observation system, which is attached to the support arm in a position near the observation system; and an operation processing unit for conducting image pickup operation and image signal processing of the image pickup unit, which is arranged on the table.

According to a sixth aspect of the present invention, in the fifth aspect of the invention, the slit lamp microscope is characterized in that the image pickup unit includes: a beam splitter for branching off light flux from an optical path of the observation system; an image pickup element for taking the image of the eye to be examined; a light flux bending member arranged opposed to the beam splitter and the image pickup element; and a condensing lens for condensing the light flux from the beam splitter onto the image pickup element through the light flux bending member.

According to a seventh aspect of the present invention, in the fifth aspect of the invention, the slit lamp microscope is characterized in that the image pickup unit includes: a beam splitter for branching off light flux from an optical path of the observation system; an image pickup element for taking an image of the eye to be examined, which is arranged opposed to the beam splitter; and a condensing lens for condensing the light flux from the beam splitter onto the image pickup element.

According to an eighth aspect of the present invention, in any one of the fifth through seventh aspects of the invention, the slit lamp microscope is characterized in that cables connecting the image pickup unit and the operation processing unit to each other is passed through a cable routing path which is led under the table by way of an accommodating groove provided in the support arm, a cable hole provided in the protruding shaft barrel portion of the base which protrudes from the pedestal to the chin rest stand side, a cable hole provided in the chin rest stand, and a cable hole provided in the table.

According to the fifth through eighth aspects of the present invention, the image pickup function with respect to the eye to be examined is improved due to the installation of the dedicated image pickup device. In addition, a slit lamp microscope can be provided in which a weight load on the support arm is reduced, the rotating operation of the observation system can be conducted in a satisfactory manner, and the cables are not exposed near the support arm so that the operability of the observation system by an examiner is excellent and also a superior outward appearance is achieved.

### BRIEF DESCRIPTION OF THE DRAWINGS

In the accompanying drawings:

FIG. 1 is a schematic side view showing a slit lamp microscope according to Embodiment 1 of the present invention;

FIG. 2 is a perspective view showing an image pickup device attached to a support arm according to Embodiment 1;

FIG. 3 is an enlarged front view showing the image pickup device attached to the support arm according to Embodiment 1;

FIG. 4 is a perspective view showing the rear side of the image pickup device according to Embodiment 1;

FIG. 5 is a schematic longitudinal sectional view showing the image pickup device attached to the support arm according to Embodiment 1;

FIG. 6 is a schematic transverse sectional view showing the image pickup device attached to the support arm according to Embodiment 1;

FIG. 7 is a partially enlarged perspective view of an operation portion of the image pickup device according to Embodiment 1;

FIG. 8 is a schematic perspective view showing a cable routing path extending from the support arm to a table according to Embodiment 1;

FIG. 9 is a perspective view indicating a cable hole which is provided in a pedestal for passing through cables according to Embodiment 1;

FIG. 10 is a schematic side view showing a slit lamp microscope according to Embodiment 2 of the present invention;

FIG. 11 is a sectional view showing an image pickup unit attached to a support arm according to Embodiment 2;

FIG. 12 is a perspective view of an operation processing unit of Embodiment 2; and

FIG. 13 is a schematic view indicating a modified example of an optical system of Embodiment 2.

### DETAILED DESCRIPTION OF THE PREFERRED EMBODIMENTS

Hereinafter, embodiments of the present invention will be described in detail with reference to the drawings.

(Embodiment 1)

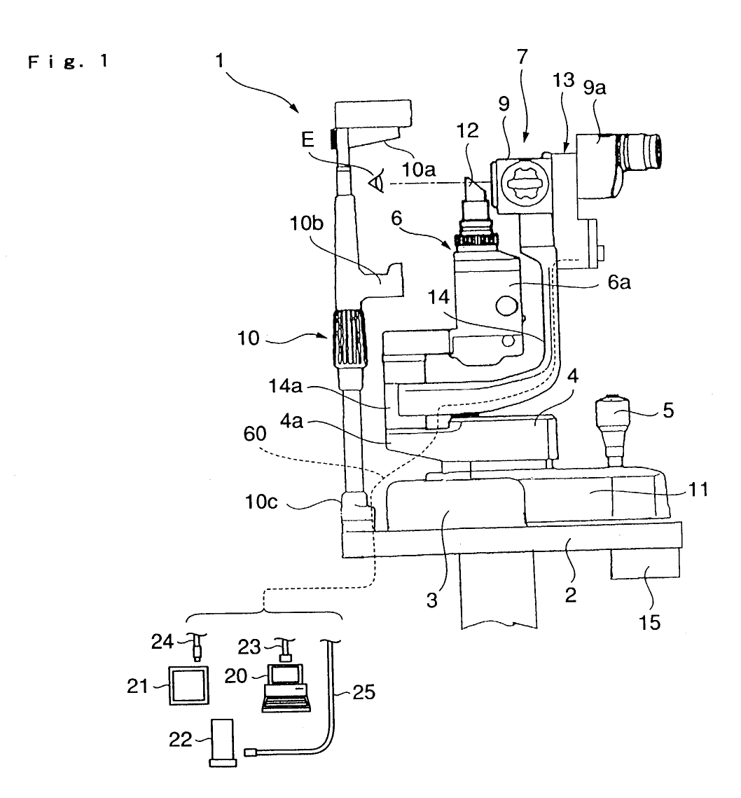

A slit lamp microscope according to Embodiment 1 of the present invention shown in FIG. 1 includes: a pedestal 11 which is supported on an ophtalmoscopic table 2 so as to be movable in horizontally lateral and longitudinal directions through a moving mechanism portion 3: a base 4 which is supported in the vertical direction by the pedestal 11; an operation handle 5 for shifting the pedestal 11 in horizontally lateral and longitudinal directions by tilting operation; an illumination system 6 having a light source portion 6a and a prism 12 which are supported so as to be rotatable in a horizontal direction by a protruding shaft barrel portion 4a of the base 4; an observation system 7 for observing an eye to be examined E; and a chin rest stand 10 having a chin rest portion 10b for the subject which is opposed to a lens barrel main body 9 including an eyepiece barrel 9a accommodating an objective lens of the observation system 7, a forehead pad 10a, and a base portion 10c attached to the table 2. The observation system 7 is supported to the upper end side of a support arm 14 which is substantially reverse L-shaped in side view and whose shaft barrel 14a in a lower end portion is axially supported by the protruding shaft barrel portion 4a of the base 4.

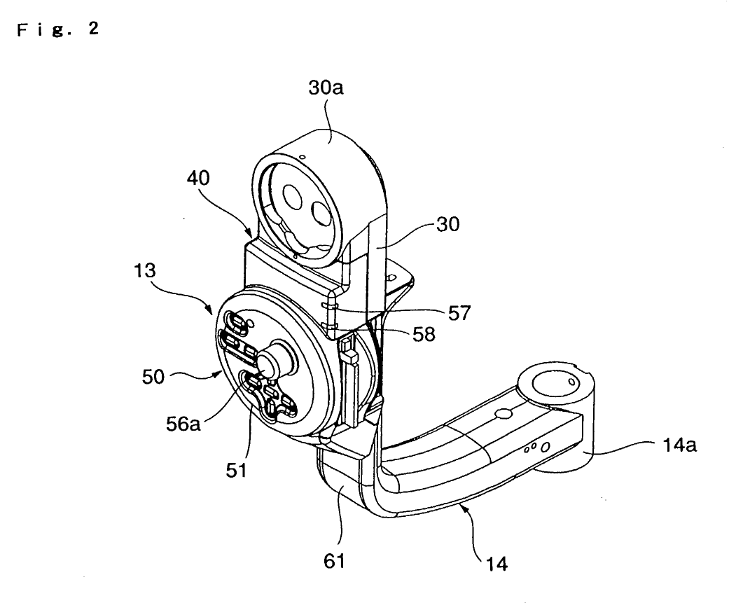

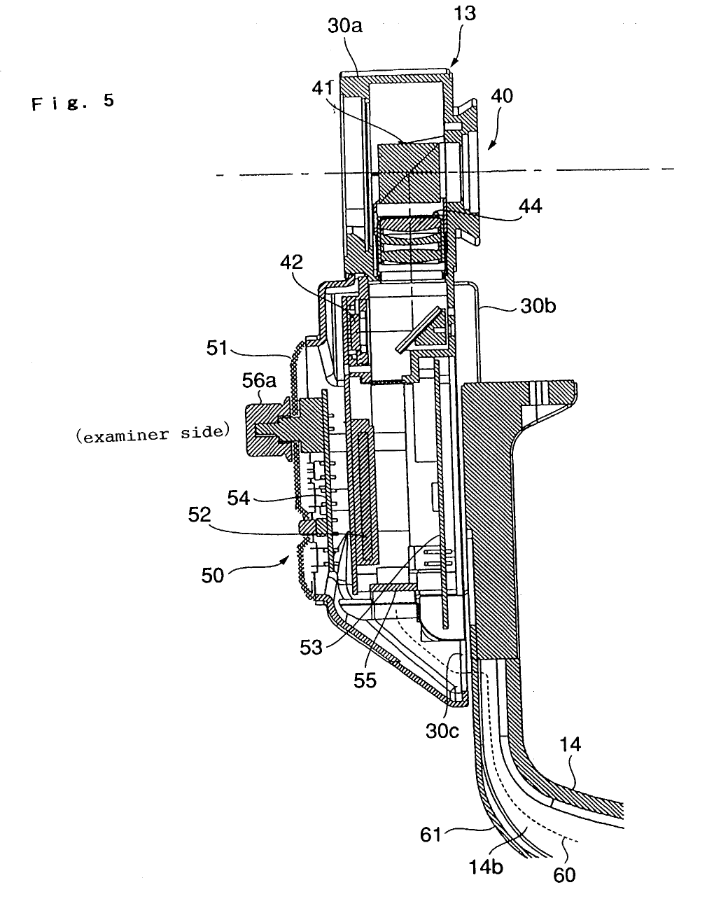

Also, an image pickup device 13 as a digital camera designed specifically for the slit lamp microscope 1 as shown in FIGS. 2, 3, 4, and 5 is attached over the lens barrel main body 9 and the front side (examiner's side) of the support arm 14.

A power source box 15 for supplying power to the light source portion 6a is arranged on the lower surface side of the table 2.

As indicated by a dotted line in FIG. 1, a cable routing path 60 for the cables which extends by way of the support arm 14, the protruding shaft barrel portion 4a of the base 4, the pedestal 11, the base portion 10c of the chin rest stand 10, and the table 2 is provided to the above slit lamp microscope 1. The cable routing path 60 will be described later in detail.

Also, in addition to the above slit lamp microscope 1, there are arranged an image processing apparatus (computer apparatus) 20 for conducting image processing on an image of an eye E to be examined, an image monitor 21 for monitoring the image of the eye E to be examined, and a power source apparatus 22 for the image pickup device 13.

The image pickup device 13 and the image processing apparatus 20 are connected with end portions of a signal transmission cable 23. The image pickup device 13 and the image monitor 21 are connected with end portions of an image monitor cable 24. The image pickup device 13 and the power source apparatus 22 are connected with end portions of a power source cable 25.

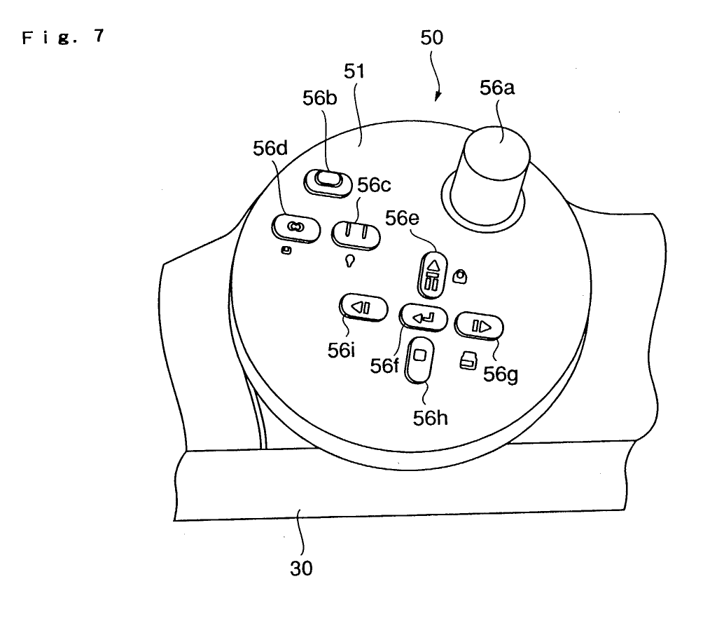

Next, the dedicated image pickup device 13 attached to the support arm 14 of the above slit lamp microscope 1 will be described in detail with reference to FIGS. 2 to 7.

The image pickup device 13 is attached to the support arm 14 in a position near the observation system 7, that is, it is arranged over the location of the eyepiece barrel 9a and the space thereunder.

The image pickup device 13 is provided with an image pickup device main body 30 in which an image pickup unit 40 and an operation processing unit 50 are integrally incorporated and which is constructed to have an approximately rectangular shape as a whole.

An optical system of the image pickup unit 40 incorporated in the image pickup device main body 30 includes: a beam splitter 41 for branching off one of light fluxes for binocular observation, which is incorporated in a connection barrel portion 30a arranged on one end side of the image pickup device main body 30 (upper side in FIGS. 2, 3, and 5) so as to face the optical path of the observation system 7; an image pickup element 42 composed of a CCD for taking an image of an eye to be examined, which is arranged in a position shifted toward the front side (examiner's side) from the optical path of the light flux branched off by the beam splitter 41; a mirror 43 for bending light flux which is opposed to the beam splitter 41 and the image pickup element 42; and a condensing lens 44 for condensing the light flux from the beam splitter 41 onto the image pickup element 42 through the mirror 43.

The operation processing unit 50 incorporated on the other end side of the image pickup device main body 30 (lower side in FIGS. 2, 3, and 5) is provided with an operation portion 51 arranged on the examiner's side and having a substantially circular shape in front view which is obtained by forming the other end side of the image pickup device main body 30 in a substantially disc shape and provided with an operation button group. In addition, on the support arm 14 side as seen from the operation portion 51, a CF card slot 52 for allowing insertion/removal of a CF (compact flash memory) card as a storage medium, an internally arranged power source board 53, an operation panel board 54 in which an IC chip for image processing and the like are mounted, and a cable connection portion 55 for connection of the cables are incorporated in the operation processing unit 50.

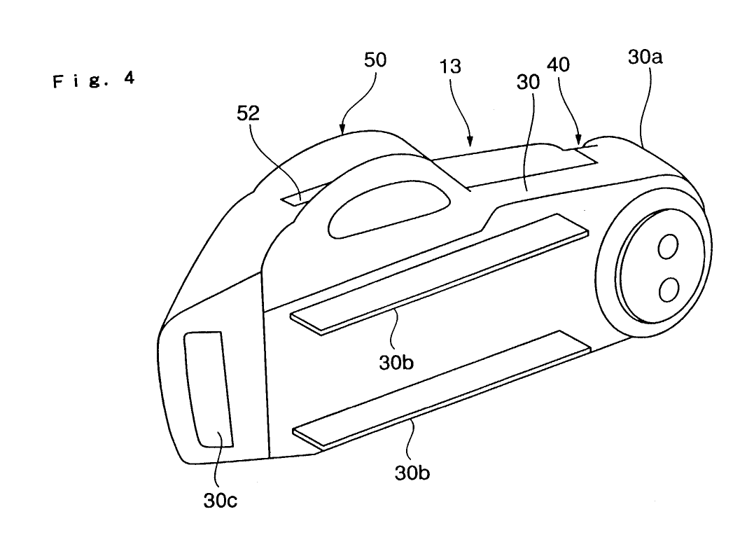

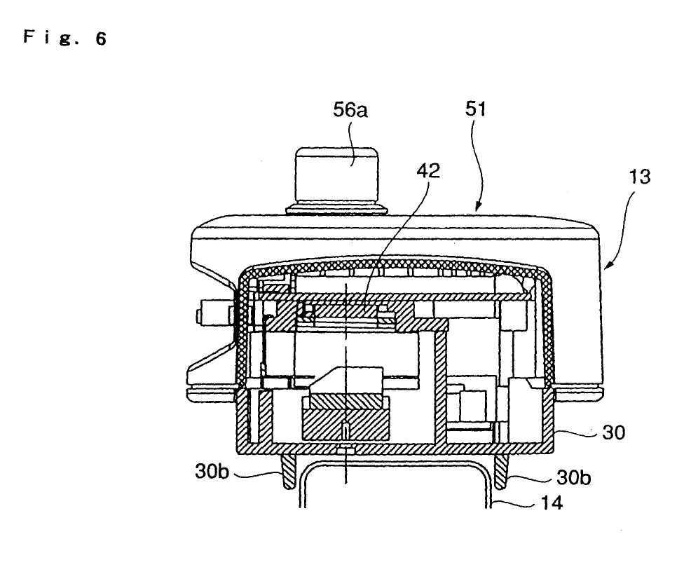

The connection barrel portion 30a of the image pickup device main body 30 is arranged between the eyepiece barrel 9a and a barrel main body portion 9b. In addition, as shown in FIGS. 4 and 6, the image pickup device main body 30 is fixed to the support arm 14 by fitting a pair of engaging pieces 30b, 30b provided in the rear side of the operation portion 51 to the support arm 14.

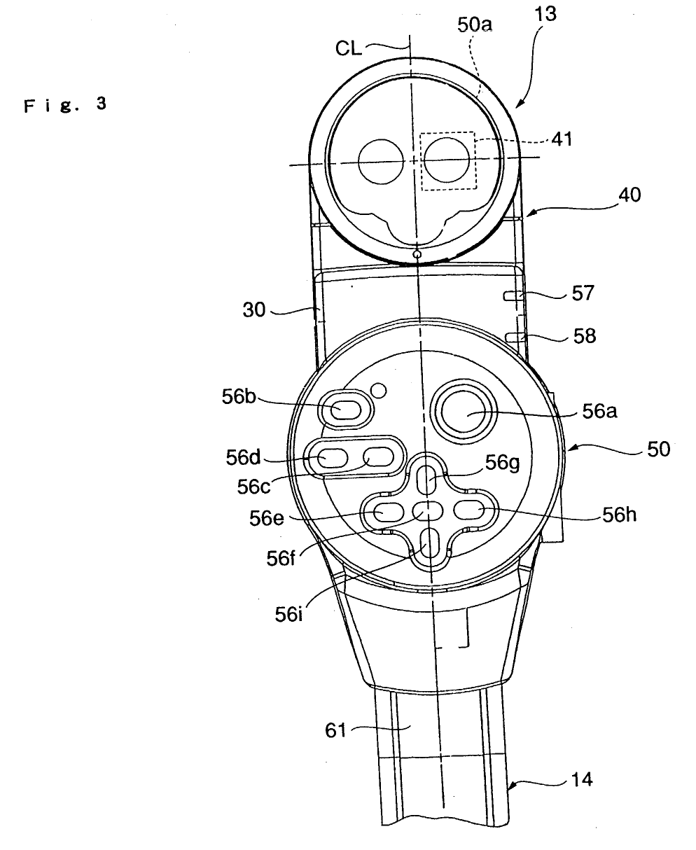

Further, as shown in FIG. 4, a cable hole 30C for passing through the cables is provided in the rear side of the operation portion 51 in the image pickup device main body 30. Note that, in FIG. 3, reference numeral 57 denotes a power source lamp and 58 denotes a CF card operation lamp.

As shown in FIGS. 2, 3, and 7, the operation button group provided to an end surface of the operation portion 51 is composed of a mode switching and on-off button 56a for switching among various image pickup modes as well as turning on and off a power source, a menu button 56b for setting various ophtalmoscopic examination menus, a first function button 56c and a second function button 56d for setting various functions, a shatter speed button 56e, a cancel button 56f, direction determination buttons 56g, 56h, and 56i, and the like. Marks serving as indexes in each operation are provided near the respective buttons of the operation button group; however, description thereof is omitted here.

In the slit lamp microscope 1 of Embodiment 1 as described above, the respective elements such as the optical systems which constitute the image pickup unit 40 and the operation processing unit 50 with the operation portion 51 of the image pickup device 13, the circuit boards, the operation button group, and the like are incorporated in the image pickup device main body 30 such that they are arranged with a good horizontal weight balance with respect to a center line CL of the support arm 14 in a vertical direction (see FIG. 3). Thus, even in a state in which the image pickup device 13 is attached to the support arm 14, the observation system 7 can be rotated about the shaft barrel 14a as a center without causing weight unbalance.

Also, the operation button group provided to the operation portion 51 of the operation processing unit 50 is attached so as to face toward the rotation center of the support arm 14, that is, the shaft barrel 14a. Further, the end surface of the operation portion 51 of the operation processing unit 50 is arranged in a direction orthogonal to the eyepiece optical axis of the observation system 7.

According to such a structure of the slit lamp microscope of Embodiment 1, due to the installation of the dedicated image pickup device 13, the image pickup function with respect to the eye to be examined is improved. In addition, even when the optical system 7 is rotated in an arbitrary direction about the shaft barrel 14a as the rotation center, an examiner looking through the eyepiece barrel 9a can, while being always opposed to the front side of the operation portion 51, cause an operating force to the operation button group to always act in the direction of the rotation center of the support arm 14. Thus, the operability of the operation portion 51 by the examiner is improved.

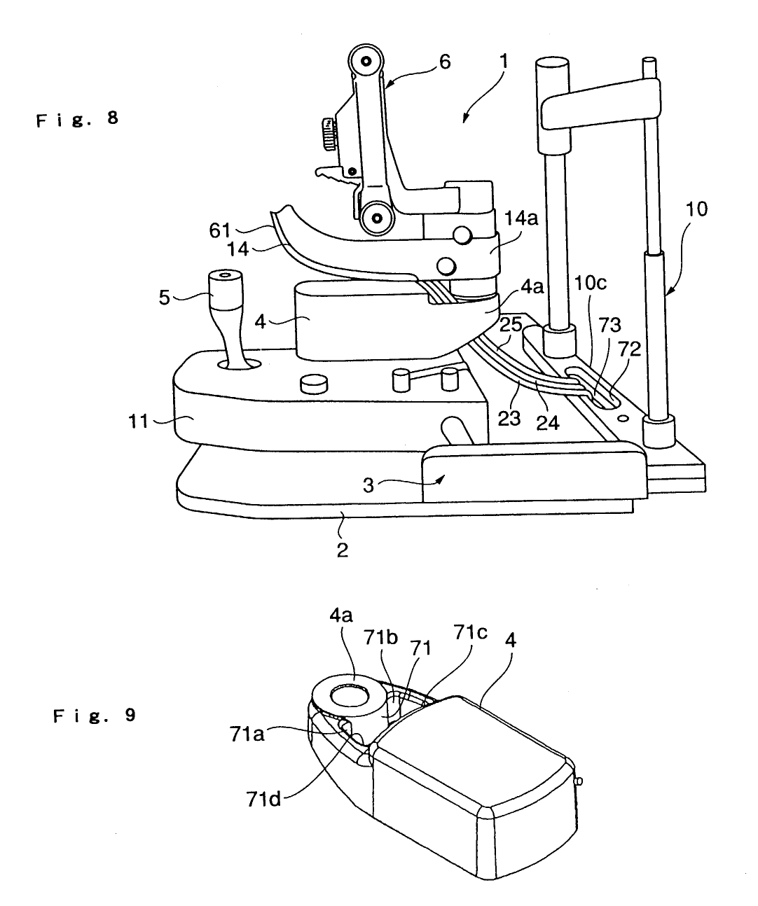

Next, the cable routing path 60 for passing through three kinds of cables consisting of the signal transmission cable 23, the image monitor cable 24, and the power source cable 25 will be described with reference to FIGS. 8 and 9.

The cable routing path 60 is constructed as follows. That is, one end portion of each of the three kinds of cables that consist of the signal transmission cable 23, the image monitor cable 24, and the power source cable 25 is connected with the cable connection portion 55 through a connector. In addition, the three kinds of cables are passed through an accommodating groove 14b with a detachable cover 61 which is provided on the rear side of the support arm 14. Then, as shown in FIG. 8, they are passed under the table 2 through a cable hole 71 provided to the protruding shaft barrel portion 4a of the base 4 which protrudes from the pedestal 11 to the chin rest stand 10 side, a cable hole 72 provided in the base portion 10c of the chin rest stand 10, and a cable hole 73 provided in the table 2. Further, the other end portions of the three kinds of cables each are connected with the image processing apparatus 20, the image monitor 21, and the power source apparatus 22, respectively.

As shown in FIG. 9, the cable hole 71 provided in the protruding shaft barrel portion 4a of the base 4 is formed as a deformed long hole which is open along the outer periphery of a barrel portion of the protruding shaft barrel portion 4a. With respect to the cable hole 71, all the four corner portions 71a, 71b, 71c, and 71d are smoothly curved. When the cables under the accommodating groove 14b are displaced according to the rotation states of the observation system 7 and the image pickup device 13 with respect to the base 4, positional regulation is effected on the cables such that damage do not occur, due to the smooth corner portions 71a to 71d, particularly, the corner portions 71a and 71b.

According to such a structure of the cable routing path 60, the cables are not exposed in the vicinity of the support arm 14 so that there can be provided the slit lamp microscope 1 which is excellent in terms of the operability of the observation system 7 by an examiner and also in terms of the outward appearance.

(Embodiment 2)

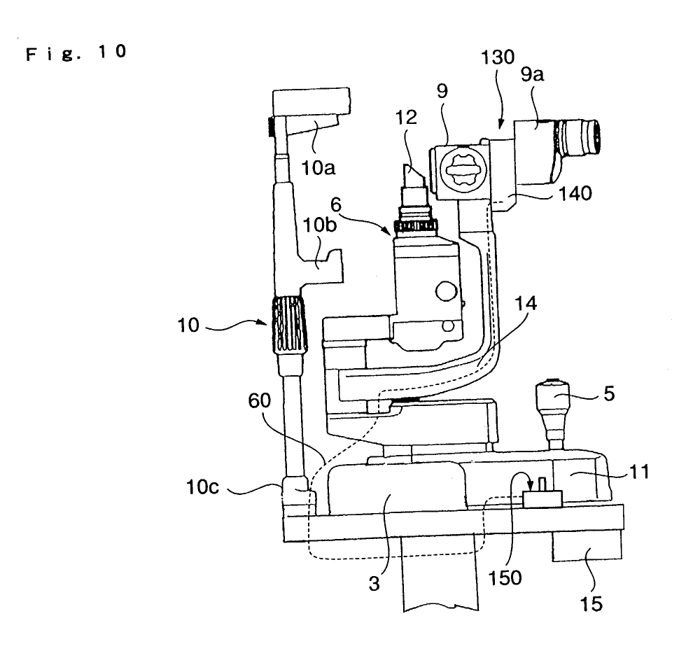

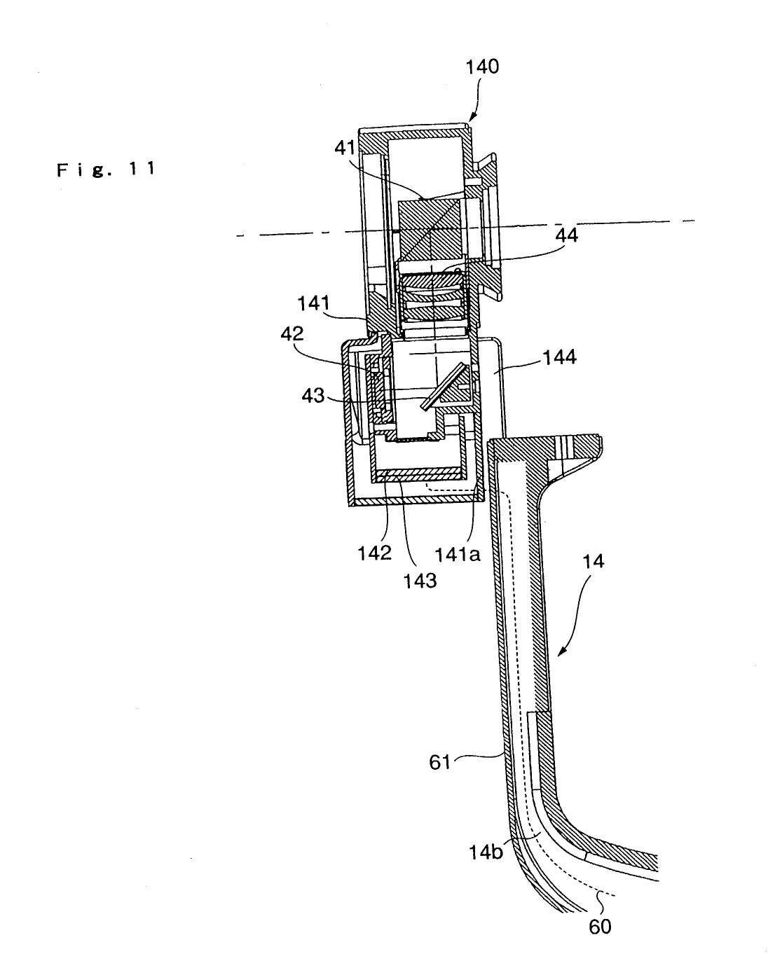

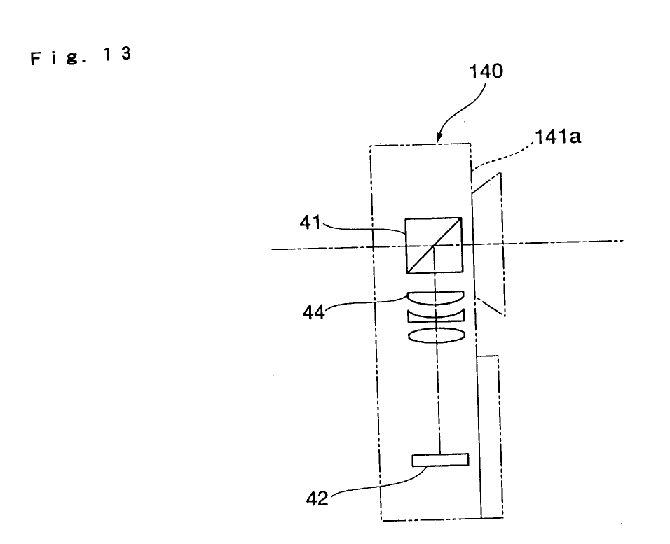

Next, Embodiment 2 of the present invention will be described with reference to FIGS. 10 to 12.

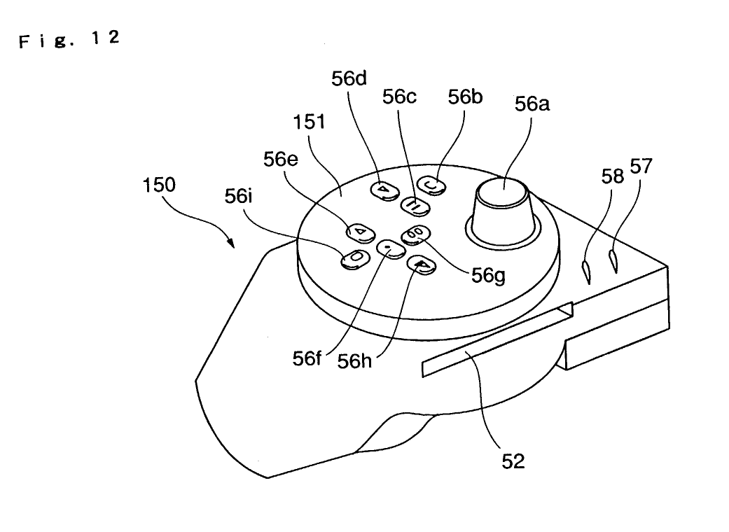

Embodiment 2 of the invention is characterized in that there is provided an image pickup device 130 specifically designed for a slit lamp microscope 1, in which an image pickup unit 140 and an operation processing unit 150 operated by an examiner are formed separately from each other. With respect to the image pickup unit 140 constituting the image pickup device 130 of Embodiment 2, as shown in FIGS. 10 and 11, a main body 141 is attached to a support arm 14 in a position near an observation system 7, that is, it is arranged over the location of the eyepiece barrel 9a and the space thereunder. In addition, the operation processing unit 150 having the same shape as the operation processing unit 50 is arranged on the table 2.

As shown in FIG. 11, an optical system of the image pickup unit 140 includes: a beam splitter 41 for branching off one of light fluxes for binocular observation, which is incorporated in a connection barrel portion 141a of the main body 140 arranged so as to face the optical path of the observation system 7; an image pickup element 42 composed of a CCD for taking an image of an eye to be examined, which is arranged in a position shifted toward the front side (examiner's side) from the optical path of the light flux branched off by the beam splitter 41 in the main body 141; a mirror 43 as a member for bending light flux, which is arranged opposed to the beam splitter 41 and the image pickup element 42; a condensing lens 44 for condensing the light flux from the beam splitter 41 onto the image pickup element 42 through the mirror 43; a signal processing circuit board 142 for processing an output signal from the image pickup element 42; and a cable connection portion 143. A cable hole 141a is provided on the rear side of the main body 141.

Also, the main body 141 is fixed to the support arm 14 with a detachable cover 61 by fitting a pair of engaging pieces 144 provided on the rear side thereof to the support arm 14.

The operation processing unit 150 is provided with an operation portion 151 arranged on the examiner's side and having a substantially circular shape in front view, which is formed in a substantially disc shape and provided with an operation button group 51a-i for image pickup operation. In addition, it is provided with a CF card slot 52 for allowing insertion and removal of a CF (compact flash memory) card as a storage medium. The structure of the operation buttons of the operation portion 151 as well as the internal structure of the operation processing unit 150 are the same as those of the above described operation processing unit 50.

The image pickup unit 140 and the operation processing unit 150 are connected with each other through the cables (signal transmission cable 23, image monitor cable 24, the power source cable 25) that are passed through the cable routing path 60 structured in the same manner as in Embodiment 1.

According to such a structure of the slit lamp microscope of Embodiment 2, due to the installation of the dedicated image pickup device 13, the image pickup function for the eye to be examined is improved. In addition, according to Embodiment 2, a construction is adopted in which only the image pickup unit 140 is attached to the support arm 14, as compared with the case of Embodiment 1. Thus, a load weight on the support arm 14 is reduced, so that the operability in rotating the observation system 7 together with the support arm 14 by the structure provided with the dedicated image pickup unit 140 can be further improved.

Note that, as shown in FIG. 13, as a modified example of the optical system of the image pickup unit 140, a structure may be adopted in which the optical system is provided with: the beam splitter 41 for branching off one of light fluxes for binocular observation, which is incorporated in the connection barrel portion 141a; the image pickup element 42 composed of a CCD for taking an image of an eye to be examined, which is arranged on the optical path of the light flux branched off by the beam splitter 41; and the condensing lens 44 for condensing the light flux from the beam splitter 41 onto the image pickup element 42. In this case, because the mirror 43 is omitted, the image of the eye to be examined which is taken by the image pickup element 42 becomes a laterally reversed image. However, it is easy to restore the laterally reversed image into a normal, unreversed image by using an image processing circuit. According to this modified example, because the mirror 43 is omitted, there is attained an advantage in that the image pickup unit 140 can be made thinner as compared with the case shown in FIG. 11, thereby achieving a reduction in size.

According to the present invention, a slit lamp microscope can be provided in which a weight load on the support arm is reduced, the rotating operation of the observation system can be conducted in a satisfactory manner, the cables are not exposed near the support arm, and the operability of the observation system by an examiner is excellent and also a superior outward appearance is achieved because the operation pressing button group is attached so as to face toward the center of the support arm.

Further, there can be provided a slit lamp microscope having a construction such that it is provided with the dedicated image pickup device having the image pickup portion and the operation processing portion formed separately from each other, whereby the image pickup function with respect to the eye to be examined is improved, the weight load on the support arm is reduced, rotating operation of the observation system can be conducted in a satisfactory manner, and the cables are not exposed in the vicinity of the support arm so that the operability of the observation system by an examiner is excellent and a superior outward appearance is achieved.

What is claimed is:

1. A slit lamp microscope comprising:
an illumination system for illuminating an eye to be examined;
an observation system for observing the eye to be examined;
a support arm for supporting the observation system on the upper end side thereof;
a base for rotatably supporting on a protruding shaft barrel portion thereof the illumination system and the lower end side of the support arm supporting the observation system;
a pedestal for supporting the base;
a chin rest stand for securing the eye to be examined with a state in which the eye to be examined is opposed to the observation system; and
a table on which the pedestal is installed and to which the chin rest stand is attached,
wherein an image pickup device composed of image pickup means for taking an observation image of the eye to be examined from the observation system and operation processing means for conducting image pickup operation and image signal processing, which has an operation portion and is integrally formed with the image pickup means, is attached to the support arm in a position near the observation system, and
an operation pressing button group is provided in a direction orthogonal to an end surface of the operation processing means along a rotation center line of the support arm.

2. A slit lamp microscope according to claim 1, wherein an end surface of the operation portion of the operation processing means is arranged in a direction orthogonal to an eyepiece optical path of the observation system.

3. A slit lamp microscope according to claim 1 or 2, wherein elements which constitute the image pickup means and the operation processing means having the operation portion of the image pickup device are incorporated in an image pickup device main body such that the elements are arranged in a balanced manner in weight with respect to a center line of the support arm.

4. A slit lamp microscope according to any one of claims 1 to 3, further comprising an accommodating groove provided in the support arm, a cable hole provided in the protruding shaft barrel portion of the base which protrudes from the pedestal to the chin rest stand side, a cable hole provided in the chin rest stand, and a cable hole provided in the table, which together form a routing path for leading cables under the table, that are to be connected with the image pickup device.

5. A slit lamp microscope comprising:
an illumination system for illuminating an eye to be examined;
an observation system for observing the eye to be examined;
a support arm for supporting the observation system on the upper end side thereof;
a base for rotatably supporting on a protruding shaft barrel portion thereof the illumination system and the lower end side of the support arm supporting the observation system;
a pedestal for supporting the base;
a chin rest stand for securing the eye to be examined with a state in which the eye to be examined is opposed to the observation system; and
a table on which the pedestal is installed and to which the chin rest stand is attached,
wherein the slit lamp microscope includes an image pickup device composed of: image pickup means for taking an observation image of the eye to be examined from the observation system, which is attached to the support arm in a position near the observation system; and operation processing means for conducting image pickup operation and image signal processing of the image pickup means, which is arranged on the table.

6. A slit lamp microscope according to claim 5, wherein the image pickup means includes: a beam splitter for branching off light flux from an optical path of the observation system; an image pickup element for taking an image of the eye to be examined; a light flux bending member arranged opposed to the beam splitter and the image pickup element; and a condensing lens for condensing the light flux from the beam splitter onto the image pickup element through the light flux bending member.

7. A slit lamp microscope according to claim 5, wherein the image pickup means includes: a beam splitter for branching off light flux from an optical path of the observation system; an image pickup element for taking an image of the eye to be examined, which is arranged opposed to the beam splitter; and a condensing lens for condensing the light flux from the beam splitter onto the image pickup element.

8. A slit lamp microscope according to any one of claims 5 to 7, wherein cables connecting the image pickup means and the operation processing means to each other is passed through a cable routing path which is led under the table by way of an accommodating groove provided in the support arm, a cable hole provided in the protruding shaft barrel portion of the base which protrudes from the pedestal to the chin rest stand side, a cable hole provided in the chin rest stand, and a cable hole provided in the table.
